(19) United States
(12) Patent Application Publication
(10) Pub. No.: US 2012/0046572 A1
(43) Pub. Date: Feb. 23, 2012

(54) Nerve stimulator measuring device

(76) Inventor: Ib Odderson, Kirkland, WA (US)

(21) Appl. No.: 12/927,508

(22) Filed: Jan. 2, 2009

Related U.S. Application Data
(63) Continuation-in-part of application No. 11/021,299, filed on Dec. 23, 2004, now Pat. No. 7,496,407.

(51) Int. Cl. A61B 5/05

(52) U.S. Cl. 600/554

(57) ABSTRACT

A nerve stimulator measuring device used to measure the length of electrical conduction of a nerve using a standard electrical nerve stimulator. The device includes a linear distance measuring device attached to the probes or the body of a standard electrical nerve stimulator. In one embodiment, the linear distance measuring device includes a housing mounted on one or both. Attached to the housing is a rotating wheel that is manually positioned over the skin and rotated as the handheld is moved over the skin. A display mounted on the housing is coupled to the wheel and use to indicate the total distance moved. The electrical nerve stimulator is then held so that the cathode probe is pressed against the skin over the nerve and adjacent to the desired distance shown on the tape. The electrical nerve stimulator is then activated and a reading is obtained.

This is a continuation-in-part application based on U.S. patent application (Ser. No. 11/021,299) filed Dec. 23, 2004 and the provisional patent applications (Ser. No. 60/532,029) filed on Dec. 23, 2003, and (Ser. No. 60/541,511) filed on Feb. 3, 2004.

### BACKGROUND OF THE INVENTION

1. Field of the Invention

This invention relates to devices used to measure nerve conduction in peripheral nerves and more particularly, to such devices that measure the conduction time and amplitude of a test signal applied to a nerve.

2. Description of the Related Art

It is common practice in medicine to measure the electrical conduction on a peripheral nerve. For example, when diagnosing carpel tunnel syndrome it is common for a physician to measure the electrical conduction in the median nerve as it extends from the forearm, through the wrist and into the hand. During the test procedure, the physician measures the length of time and the amplitude of a test signal applied to the nerve having a known length. To perform the test, recording sensors are attached to the patient's forearm and a nerve stimulator is positioned over the nerve.

When testing for carpel tunnel syndrome, the recording sensors and the nerve stimulator's cathode probe must be spaced apart at selected distances (8 cm, 10 cm, and 14 cm) on the hand and forearm. Heretofore, physicians have used a ruler or measuring tape and an ink marker to first mark the specific locations of the recording electrodes and the nerve stimulator on the patient's skin before the test is performed. Often, several tests are performed on the same hand and forearm during the visit, which requires manually marking the skin reference points. The act of measuring and marking several sets of reference points on the forearm and hand is very time consuming. Also, because the sets of reference points are relatively close, a wrong set of reference points may be used during the test that produces inaccurate readings.

### SUMMARY OF THE INVENTION

It is an object of the present invention to provide a nerve stimulator measuring device that enables a physician to easily and quickly determine the proper position of the nerve stimulator.

It is another object of the invention to provide such a device that may be used with a standard electrical nerve stimulator that uses a cathode probe and an anode probe that are positioned against or adjacent to the skin.

It is another object of the invention to provide such a device that enables a physician to determine different locations of the cathode probe from the electrical sensor without using an ink marker.

It is another object of the invention to provide such a device that is wireless thereby eliminating wires that typically extend from the device to the recording machine.

These and other objects are met by the nerve stimulator measuring device with a tape measure attached thereto used to measure the distance between the electrical sensor and the cathode probe. In the first embodiment, the tape measure is located in an outer housing that attaches or is integrally formed on the cathode probe on a standard electrical nerve stimulator. The outer housing includes two bores designed to receive the anode and cathode probes on the electrical nerve stimulator. During assembly, the outer housing is positioned over the two probes with the tape measure disposed therebetween. An index marking or line formed on the outer surface of the outer housing is aligned with the center axis on the cathode probe.

In a second embodiment, the nerve stimulator comprises an outer housing with a tape receiver cavity formed there that holds a spool upon which a flexible tape measure is wound and unwound. The spool is coupled to a tape retraction mechanism that automatically rewinds the tape measure on the spool. Mounted on the outer surface of the outer housing is a stimulator activation button coupled to an electric test signal generator and a tape retraction button coupled to the tape retraction mechanism.

In the second embodiment, disposed on the distal end of the tape measure used with the second embodiment, are three recording sensors. Wires extend from the three recording sensors to an optional wireless transmitter located inside the outer housing. During operation, the wireless transmitter transmits the detected electrical signal information from the sensors to a wireless receiver connected to a nearby recording machine. The three wires that connect to the three recording sensors are mounted on the tape measure and are extended and retracted into the outer housing with the tape measure. Also mounted on the outer housing is a signal intensity control switch that the user manually operates to adjust the size of the signal generated by the stimulator probes.

In three other embodiments of the invention, a linear skin distance measuring device is attached to the electrical nerve stimulator. In two embodiments, a linear skin distance measuring device is attached to one or both probes on the electrical nerve stimulator. In another embodiment, the linear skin distance measuring device is attached to the body of the electrical nerve stimulator. In each embodiment, the linear skin distance measuring device is designed to measure the distance the electrical nerve stimulator travels moved to a desired location on the skin over the nerve to be tested from an electrode sensor attached to the skin. An electric nerve generator is connected to the anode and cathode probes on the electrical nerve stimulator. The electrical nerve stimulator is positioned over the electrode sensor and then manually moved to the desired location over the nerve. A display on the device informs the healthcare worker the precise distance traveled. When the desired distance is achieved, the test is then performed.

When the first embodiment is used to diagnose carpel tunnel syndrome, the recording sensors are first attached to the forearm over the median nerve. The free end of the tape measure is then centrally aligned over the first recording sensor and the electrical stimulator with the outer housing attached thereto is pulled towards the hand to the desired length (8 cm, 10 cm, or 14 cm) required for the test. The electrical nerve stimulator is then held so that the cathode probe is aligned on the skin adjacent to the desired distance on the tape. The electrical nerve stimulator is then activated and a reading is obtained. When additional tests are to be conducted, the recording sensor is again used as a reference point for the free end of the tape. The electric nerve stimulator is moved to the new testing point so that the desired distance is displayed on the tape. The electrical nerve stimulator is then held so that the cathode probe is then pressed against the skin adjacent to the new distance.

When the second embodiment is used to diagnose carpel tunnel syndrome, the end of the tape measure is pulled from the outer housing so that the three electrical sensors are longitudinally aligned at a desired location of a desired nerve on the forearm. The outer housing is then pulled towards the hand so that the anode and cathode stimulator prongs are positioned at a desired location. (8 cm, 10 cm, or 14 cm) on the tape measure. The stimulator button is then pressed to activate the electrical nerve stimulator. The optional signal intensity switch is used to adjust the desired signal intensity. When additional tests are to be conducted, the nerve sensor probes are moved to a new location on the tape measure and the stimulator button is activated. When the test is completed the tape retraction button is activated to automatically retract the tape measure into the outer housing.

In the third and fourth embodiments, the handheld electrical nerve stimulator is perpendicularly aligned over the skin adjacent to an electrode sensor. The distance measuring device is then activated and begins to measure the distance the handheld electrical nerve stimulator is moved over the surface of the skin. When the handheld electrical nerve stimulator is positioned at the desired location on the skin, the distance reading on the display is then recorded and the two probes are then pressed the skin. The electric nerve generator is then activated and a test is then conducted.

### DESCRIPTION OF THE DRAWINGS

FIG. 1 is a perspective view of the first embodiment of the nerve stimulator measuring device disclosed herein.

FIG. 2 is a side elevational view of another embodiment of the nerve stimulator measuring device.

FIG. 3 is a top plan view of the first embodiment of the nerve stimulator measuring device shown in FIG. 1.

FIG. 4 is a side elevational view of the first embodiment of the nerve stimulator measuring device shown in FIGS. 1 and 3.

FIG. 5 is a top plan view of the second embodiment of the nerve stimulator measuring device shown in FIG. 2.

FIG. 6 is a side elevational view of the second embodiment of the nerve stimulator measuring shown in FIGS. 2 and 5.

FIG. 7 is a perspective view of a third embodiment of the nerve stimulator measuring device used with a magnetic nerve stimulator.

FIG. 8 is a second perspective view of the third embodiment of an electromagnetic nerve stimulator measuring device shown in FIG. 7.

FIG. 9 is a perspective view of two tab sensors directly connected to the distal end of tape 30.

FIG. 10 is a perspective view of the distal end of the tape with two wrap sensors attached thereto.

FIG. 11 is an illustration showing a fourth embodiment of the nerve stimulator measuring device used to measure the conductivity of a nerve on a patient's hand.

FIG. 12 is a top plan view of the fourth embodiment of a nerve stimulator measuring device.

FIG. 13 is a right side elevational view of the fourth embodiment of the nerve stimulator measuring device shown in FIGS. 11 and 12.

FIG. 14 is a left side elevational view of the nerve stimulator measuring device shown in FIGS. 11-13.

FIG. 15 is a front elevational view of the nerve stimulator measuring device shown FIGS. 11-14.

FIG. 16 is a rear elevational view of the nerve stimulator measuring shown in FIGS. 11-15.

FIG. 17 is a top plan view of a third embodiment of the tape measure.

FIG. 18 is a sectional view taken along line 18-18 FIG. 17.

FIG. 19 is a perspective view of tape measure shown in FIGS. 17 and 18 rolled onto a spool.

FIG. 20 is a front elevational view of a handheld electrical nerve stimulator with a linear distance measuring device mounted on the lower end of the stimulator's body with a lower platform that slides up and down over the anode and cathode probes.

FIG. 21 is a side elevational view of the measure device shown in FIG. 20.

FIG. 22 is a top plan view of the measure device shown in FIGS. 20 and 21.

FIG. 23 is a side elevational view of another embodiment of a linear distance measuring device mounted on the end of the nerve stimulator with a lower platform that slides over one probe on the electrical nerve stimulator.

FIG. 24 is a top plan view of the embodiment shown in FIG. 23.

FIG. 25 is a side elevational of another embodiment of the nerve stimulator with an optical linear measuring unit built therein.

FIG. 26 is another embodiment of the nerve stimulator with an optical linear measuring unit that uses a roller ball to measure the distance traveled over a surface.

### DESCRIPTION OF THE PREFERRED EMBODIMENT(S)

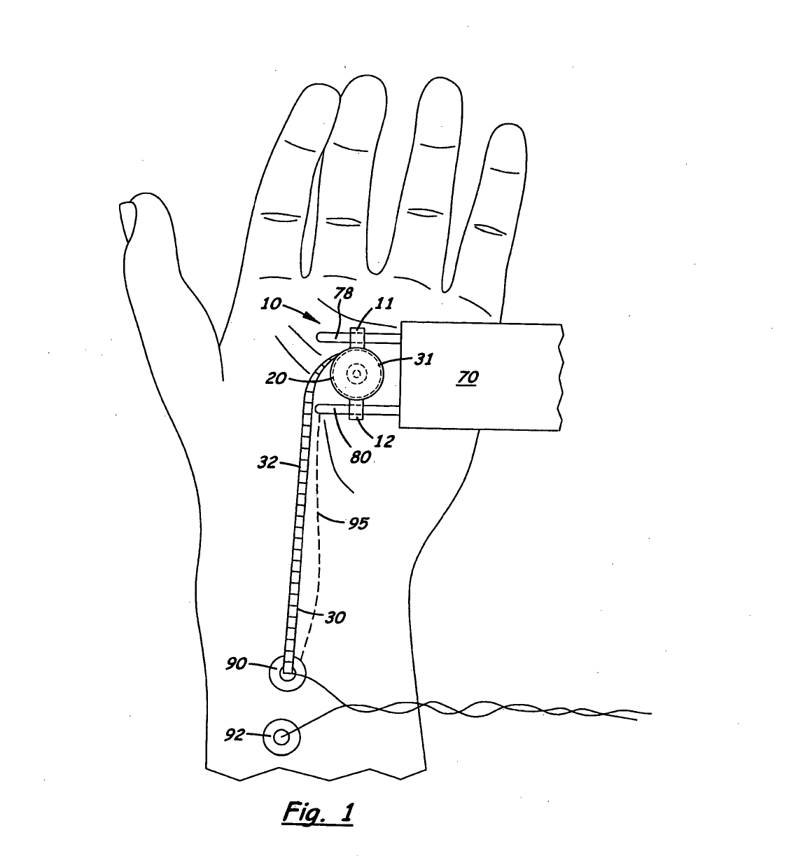

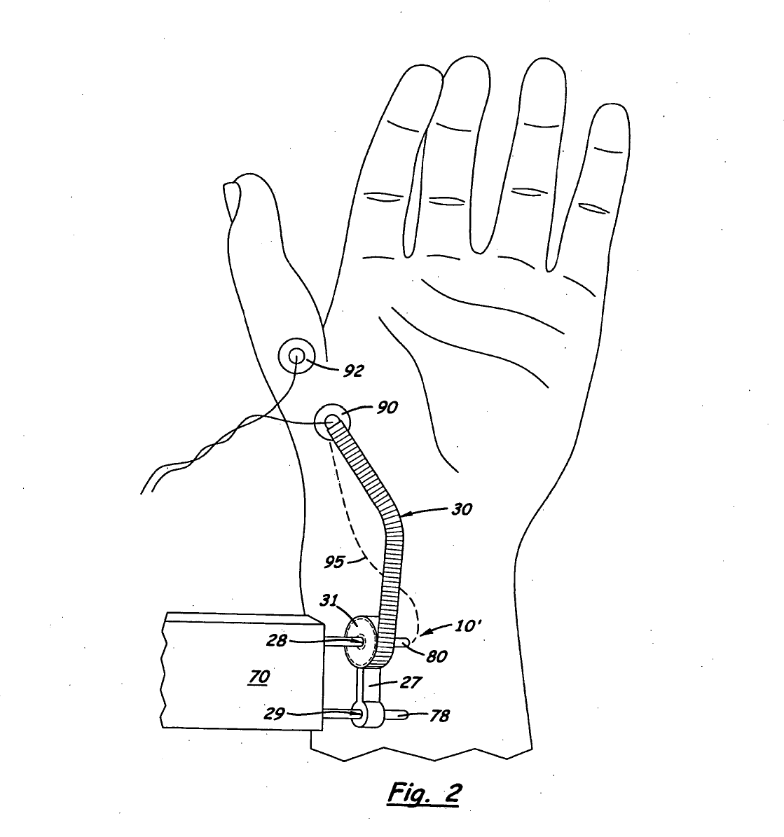

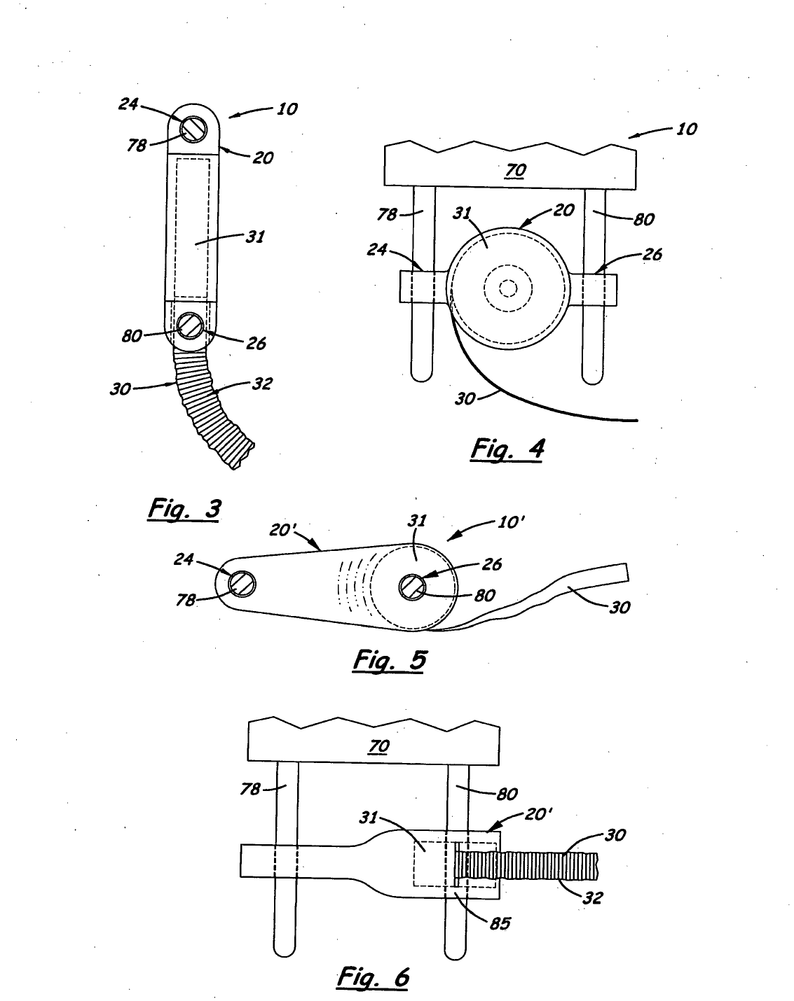

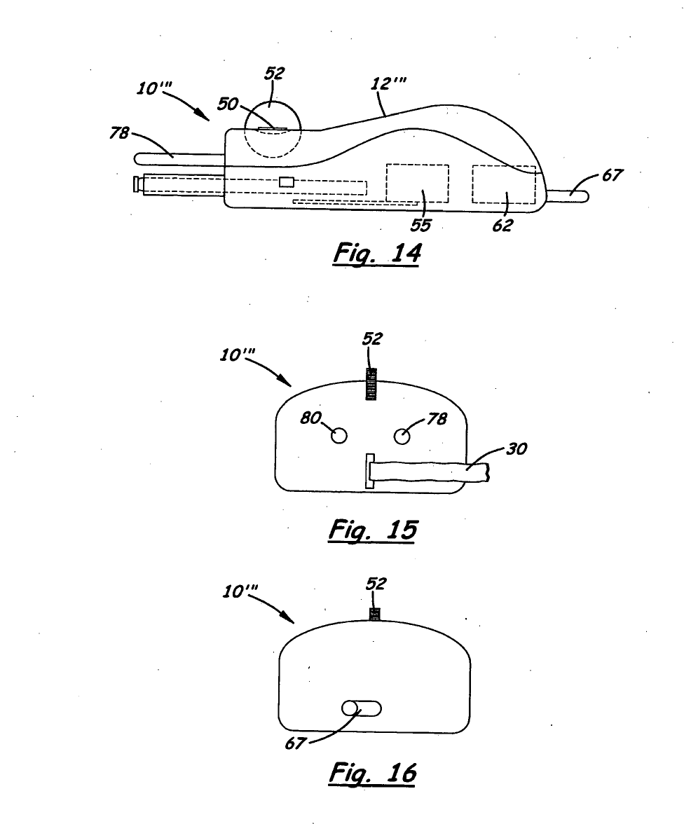

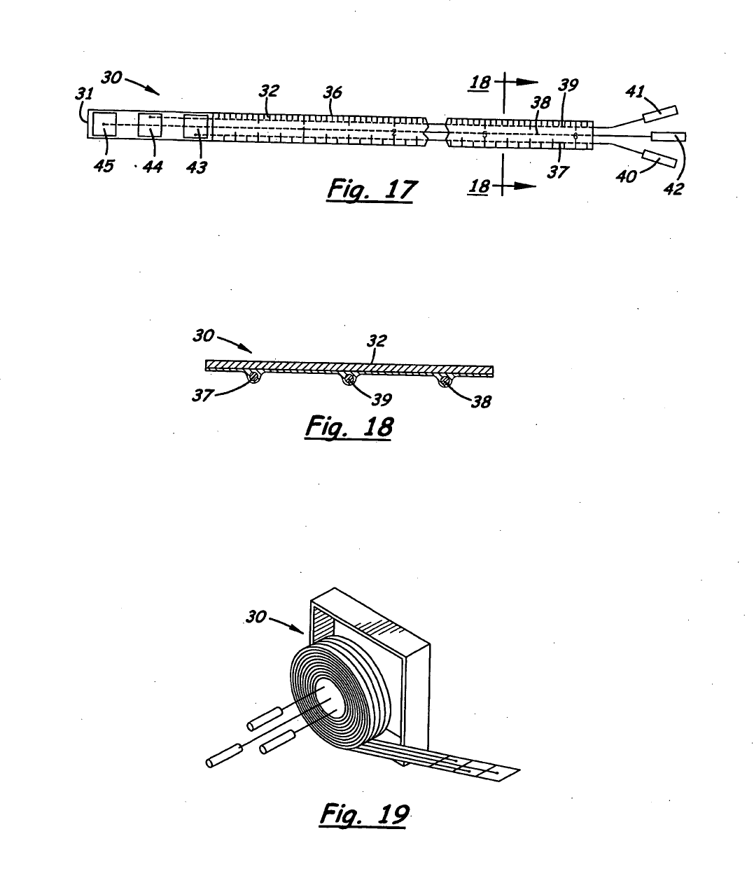

Shown in the accompanying FIGS. 1-26 are six embodiments of an electrical nerve stimulator measuring device used to measure the distance of conductivity in a peripheral nerve. Referring to the first embodiment shown in FIGS. 1, 3, and 4, the device 10 comprises an outer housing 20 with two side ears 11, 12 that attach to the anode and cathode probes 78, 80, respectively, on a handheld electrical nerve stimulator 70.

Located inside the outer housing 20 is a retractable spool 31 with a flexible tape 30 with length measure units 32 printed thereon. In the preferred embodiment, the two ears 11, 12 include two bores 24, 26 designed to slidingly receive the anode and cathode probes, 78, 80 respectively. The outer housing 20 is aligned on the probes 78, 80 so that the tape measure 30 unwinds around a center axis that is perpendicular to the longitudinal axis of the two probes 78, 80.

The second embodiment of the device 10′, shown in FIGS. 2, 5 and 6, comprises the tape measure 30 also disposed inside an outer housing 20′ designed to be coaxially aligned around the cathode probe 80. The outer housing 20′ includes a center bore 28 that receives the cathode probe 80 on the electrical nerve stimulator 70. A portion 27 of the outer housing 20′ extends laterally and includes a second bore 29 designed to slidingly receive the anode probe 78. The spool 31 for the tape measure 30 is aligned inside the outer housing 20′ so that it unwinds around a center axis coaxially aligned with the cathode probe 80. When properly assembled on the electrical nerve stimulator 70, the anode probe 78 extends through the second bore 29 and prevents the outer housing 20′ from rotating on the stimulator 70.

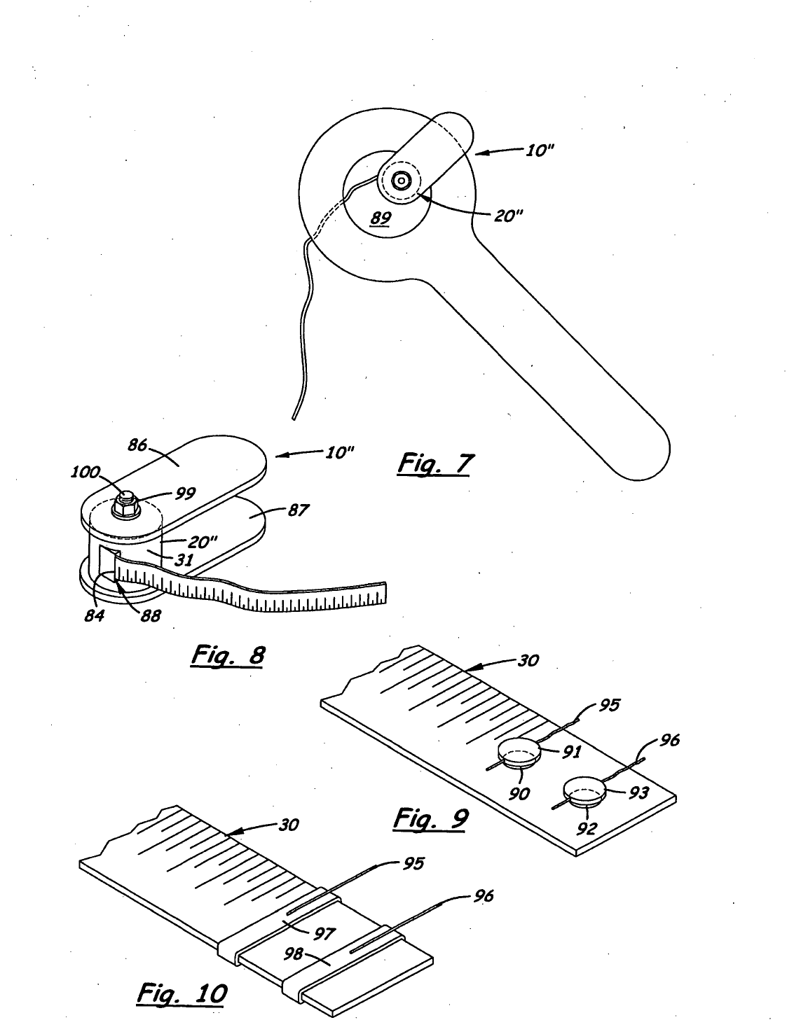

FIGS. 7 and 8 show a third embodiment of the measuring device, denoted 10″, design to be used with an electro-magnetic nerve stimulator 85. Device 10″ comprises two clamping members 86, 87 located on the opposite sides of a cylindrical shaped outer housing 20″. Like the first two embodiments, located inside the outer housing 20″ is a retractable spool 31 with a flexible tape measure 30 wound thereon. Formed on the side of the outer housing 20″ is an exit port 88 through which the distal end of the tape measures 30 extends. The two clamping members 86, 87 are designed to extend and adjustably squeeze around the circular body of the electrical nerve stimulator 85. A threaded bolt 100 and nut 99 are used to apply a clamping force to the two clamping members 86, 87. The outer housing 20 is aligned on the two clamping members 86, 87 to that its center axis is perpendicular to the longitudinal axis on the two clamping members 86, 87. When properly assembled, the exit port 88 is aligned over the center axis of the center opening 89 on the electrical nerve stimulator 85.

In the first three embodiments 10, 10′, 10″, an optional index marking or surface 84 may be printed or formed on the outer body 20, 20′, or 20″ that denotes the reference point for the tape measure 30.

As shown in FIGS. 1 and 2, during use, the two recording sensors 90, 92 are positioned on the skin over or proximal end of the nerve 95. The end of the tape measure 30 is then grasped and aligned with the center axis of the recording sensor 90, 92. The electrical nerve stimulator 70 is then pulled toward the hand to unwind the tape measure 30 from the outer housing 20, 20′. Using the index mark on the outer housing 20 and the length measurement units 32 on the tape measure 30, the electrical nerve stimulator 70 is then positioned so that its cathode probe 80 is placed at the desired location on the skin over the nerve 95 and adjacent to the desired distance shown on the tape measure 30. The electrical nerve stimulator 70 is then activated and a reading is obtained. When additional tests are to be conducted, the first recording sensor 90 is used as a reference point, and the tape measure 30 unwound from the outer housing 20 until the desired length is indicated. The electrical nerve stimulator 70 is then selected and the cathode probe 80 is then aligned over the skin adjacent to the new desired distance.

When using the third embodiment of the device 10″, the electro-magnetic stimulator 85 is held so that the center axis of the central opening 89 is longitudinally aligned over the nerve 95. The stimulator 85 is held so that the exit port 88 of the device 10″ is positioned directly over the nerve 95. The end of the tape measure 30 is then pulled and positioned over the sensor. The distance indicia on the tape measure 30 at the exit port 88 or surface 84 is then read. With devices 10, 10′ and 10″, the recording sensors 90 and 92 may be attached or formed in the distal end of the tape measure 30. As shown in FIG. 9, the recording sensors 90, 92 may be “button-like” tab connectors 91, 93, respectively, that connect to the lead wires 95, 96 that connect to the recording machine. In FIG. 10, the connectors 91, 93 are replaced with two strap connectors 97, 98, respectively, that wrap around tape measure 30 and connect to lead wires 95, 98, respectfully.

Shown in the accompanying FIGS. 11-18, is a fourth embodiment of the device, denoted 10′″ also used to measure the distance of conductivity in a peripheral nerve 95. Referring to FIGS. 11, 13, and 14, the device 10′″ comprises an outer housing 20′″ with anode and cathode probes 78, 80, respectively, are longitudinally aligned and extending from one end. Mounted inside the outer housing 20′″ is a tape measure receiver cavity 35 that holds a spool 37 and a tape retraction mechanism (not shown) that automatically rewinds the tape measure 30 onto the spool 37.

The three recording sensors 43, 44, 45 are mounted longitudinally near the distal end 31 of the tape measure 30. Printed on the front surface 32 of the tape measure 30 are metric or English distance markings 36 that enable the user to determine the distance from the closest recording sensor. Also mounted on the outer surface of the outer housing 20′″ is a tape retraction button 65 coupled to the tape retraction mechanism 39 which when activated, automatically retracts the tape measure 30 into the outer housing 12.

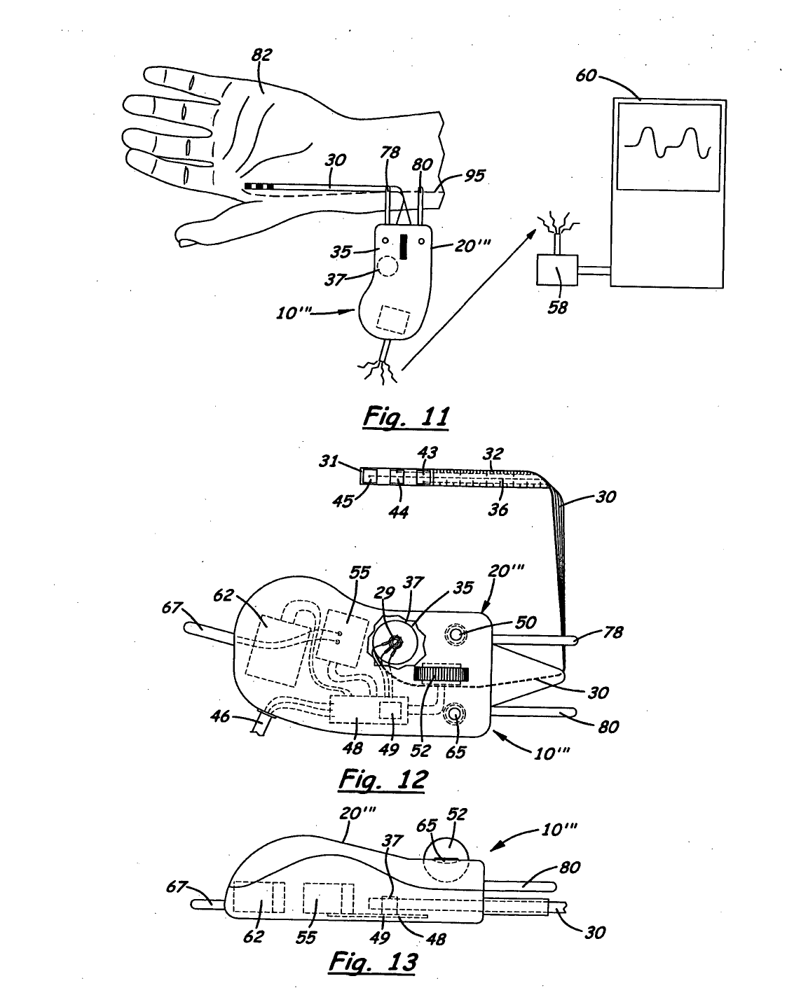

As shown in FIG. 12, an electric test signal generator 49 is mounted on a printed circuit board 48 disposed inside the outer housing 12′″. Wires from the two prong stimulators 78, 80 connect to a printed circuit board 48. During operation, the test signal generator 49 produces a test signal to the two prong stimulators 78, 80. It should be understood however, that the electrical test signal generator 49 may be eliminated from the outer housing 20′″ and mounted in an external device (not shown) that is connected to the outer housing 20′″ via a cable 67. Also mounted on the outer surface of the outer housing 20′″ is a stimulator activation button 50 coupled to the electric test signal generator 49. A test single intensity dial 52 is also provided to allow the user to adjust the intensity of the test signal.

Located inside the outer housing 20′″ is an optional wireless transmitter 55 connected printed circuit board 48. During operation, the wireless transmitter 55 transmits detected electrical signal information from three sensors 43, 44, 45 to a wireless receiver 58 connected to a nearby recording machine 60 shown in FIG. 11. When the wireless transmitter 55 is not provided in the device, the three wires connect directly to main cable 46 that runs to the recording machine 60. Located inside the outer housing 20′″ is a 9 volt battery 62 that provides electricity to the probes 78, 80 and to the printed circuit board 48

When device 10′″ is used to diagnose carpel tunnel syndrome, the distal end 31 of the tape measure 30 is pulled from the outer housing 12 so that the three electrical sensors 43, 44, 45 are aligned at the desired location on the hand 82. The outer housing 20′″ is then pulled so that the anode and cathode stimulator prongs 78, 80 are positioned at a desired location (8 cm, 10 cm, or 14 cm) on the tape measure 30 along the forearm. The stimulator button 50 is then pressed to activate the electrical test signal generator 49. The optional signal intensity dial 52 is used to adjust the signal intensity. When additional tests are to be conducted, the nerve sensor probes 78, 80 are moved to a new location on the tape measure 30 and the stimulator button 50 is activated. When the test is completed the tape retraction button 65 is activated to automatically retract the tape measure 30 into the outer housing 20′″.

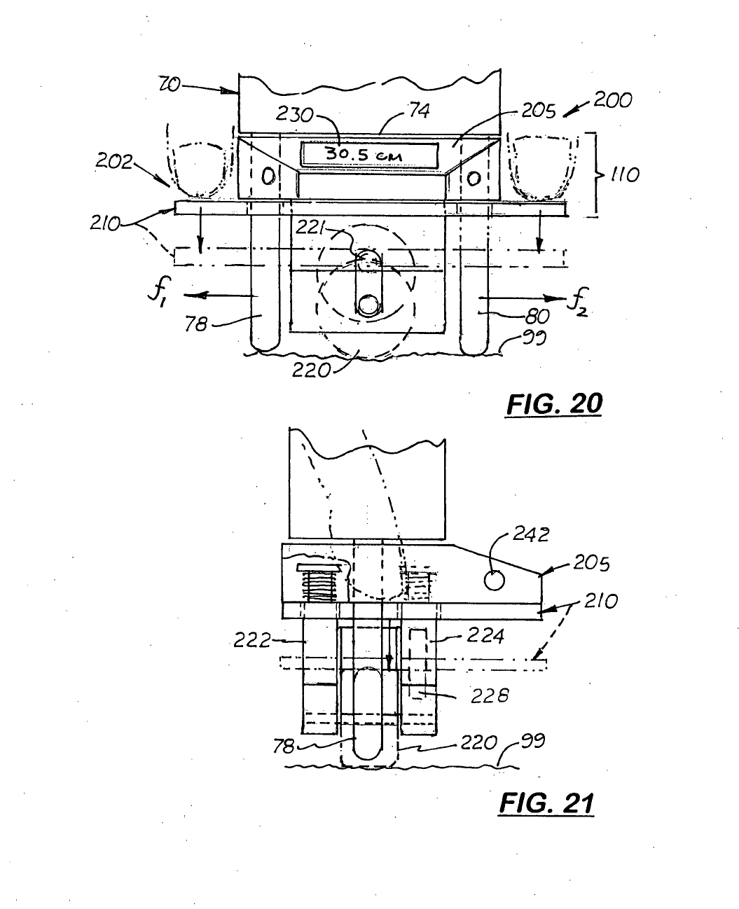

FIGS. 20-24 show two additional embodiments of the invention denoted 200, 300 in which a manual linear distance measuring device 202, 302 is selectively attached or integrally mounted on the end of the electrical nerve stimulator 70. In FIGS. 20-22, the linear skin distance measuring device 202 includes a main body 205 that securely attached to the upper ends of one or both probes 78, 80 or to the nerve stimulator 70. Located below the main body 205 is a moveable lower platform 210 with a rotating wheel 220 mounted on an axle 221 designed to roll over the surface of the skin 99. Coupled to the rotating wheel 220 and mounted on the main body 205 is an indicator or display 230 that informs the healthcare worker the linear distance traveled by the rotating wheel 220 during use.

The lower platform 210 includes two bores 212, 214, designed to slide over the two probes 78, 80, respectively. The rotating wheel 220 is mounted on an axle 221 held between two, transversely aligned, rigid supports 222, 224 that extend downward from the lower platform 210. A transducer 228 is provided for converting the rotational movement into a digital format. The two rigid supports 222, 224 are parallel and spaced apart so that the rotating wheel 220 may rotate freely between them. The rigid supports 222, 224 are also slightly shorter than the diameter of the rotating wheel 220 so that the two supports 222, 224 are above the skin 99 as the lower surface of the rotating wheel 220 contacts and rotate over the skin 99. During use, the rotating wheel 220 rolls over the skin surface when the nerve stimulator 70 is moved laterally (directions f1 and f2) as shown in FIG. 20.

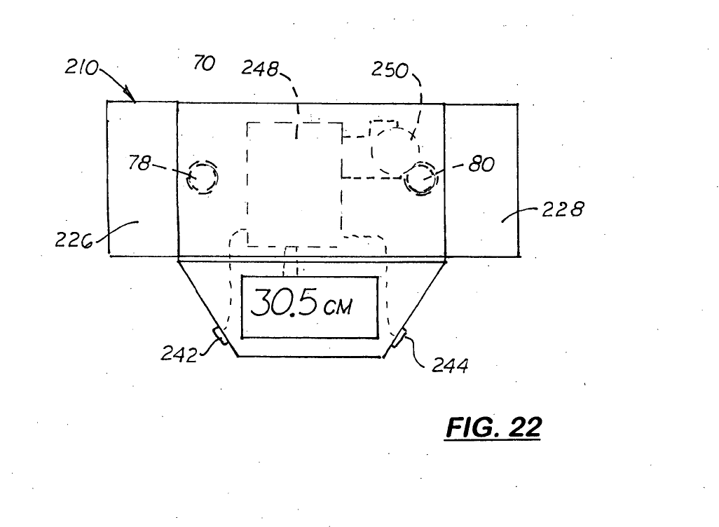

As shown in FIG. 22, the lower platform 210 includes two lateral ears 226, 228 which the healthcare worker presses against using his or her finger to force the rotating wheel 220 against the skin 99. During use, the lower platform 220 is force downward over the two probes 78, 80 to press the rotating wheel 220 against the skin 99 as the nerve stimulator 70 is moved laterally to the designed skin position over the skin 99

In the preferred embodiment, the rotating wheel 220 is biased upward towards the main body 205 when not in use thereby enabling the nerve stimulator 70 to be used in a normally manner without the linear distance measuring device 200. Attached to the two support arms 222, 224 are two t-shaped posts, 225, 227, respectively, that extend vertically upward and into a void space created inside the main body 205. Springs 236, 238 are attached to the two posts 225, 227, respectively, which press against the inside surface of the main body 205 to biased the lower platform 210 upward.

FIG. 22 is a top plan view of the measure device shown in FIGS. 20 and 21 with a LCD display 230 mounted on the front surface of the main body 205. Mounted on the sides of the main body 205 is a ON/OFF switch 242 and a RESET switch 244. The display 230 and the two switches 242, 244 are connected to a PCB 248 mounted inside the main body 205. A battery 250 is mounted inside the main body 205 and electrically connected to the PCB 248.

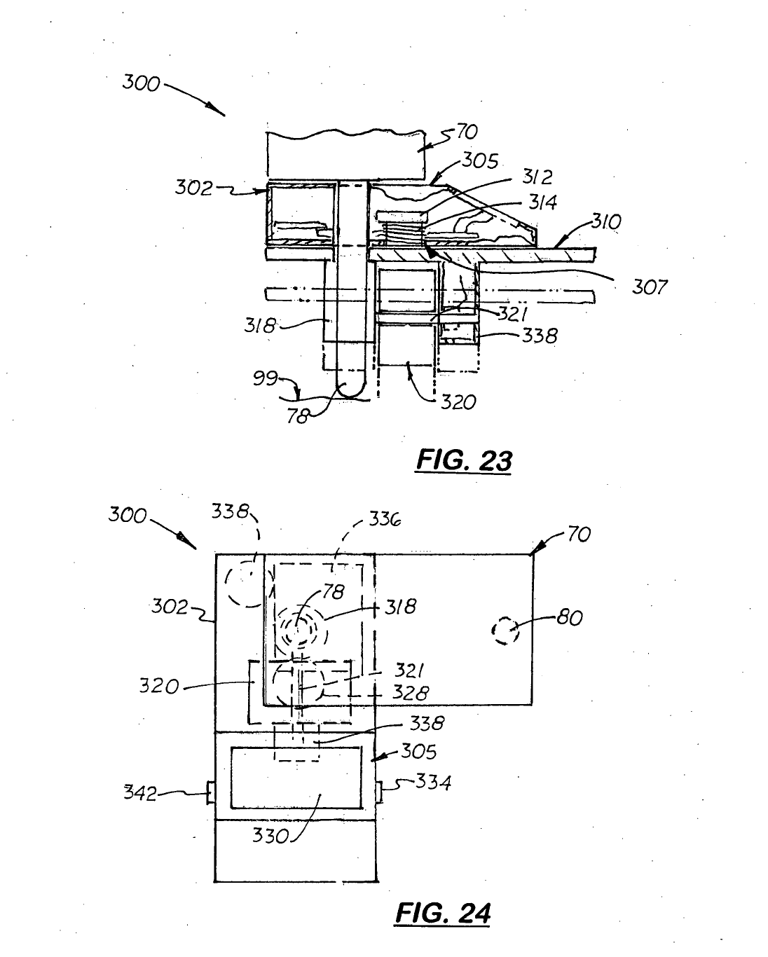

FIG. 23 is a side elevational view of the nerve stimulator 70 with another embodiment of a linear distance measuring device, denoted 302, with the main body 305 mounted on the upper ends of one of the two probes 78, 80 and the lower platform 310 that slides up and down over one probe 78 or 80. Mounted on the lower platform 310 is a T-shaped post 312 that extends into the void cavity formed in the main body 305. A spring 314 is positioned around the post 312 which extends through a bore 307 formed on the bottom surface of the main body 50. During use, the spring 314 presses against the inside surface of the main body 305 and acts as a biasing means to hold the lower platform 310 upward over the probe 78 when not in use.

The main body 305 includes display 330, a PCB 336, a battery 338 and an ON/OFF switch 342 and a RESET switch 344. The lower platform 310 includes a rear cylindrical member 318 that slides over one probe 78 or 80. Located in front of the cylindrical member 318 is a rigid support member 338. A rotating wheel 320 is mounted on an axle 321 and inside the space created between the cylindrical member 318 and the rigid member 338. Located inside is a transducer 328 used to convert rotational movement into a digital format.

The lower platform 310 is sufficiently wide and long so that a portion of the lower platform 310 extends laterally and forward to the main body 305 and exposed. The exposed portions may be used as pressing surfaces for the user's finger tips to press the lower platform 310 and the rotating wheel 320 when moving the nerve stimulator 70 into a desired location.

In each embodiment, the linear skin distance measuring device 200, 300 is designed to measure the distance the electrical nerve stimulator travels moved to a desired location on the skin over the nerve to be tested from an electrode sensor attached to the skin 99. An electric nerve generator is connected to the anode and cathode probes on the electrical nerve stimulator 70. The electrical nerve stimulator is positioned over the electrode sensor and then manually moved to the desired location over the nerve. A display 230, 330 on the device 200 or 300, respectively, informs the healthcare worker the precise distance traveled. When the desired distance is achieved, the test is then performed.

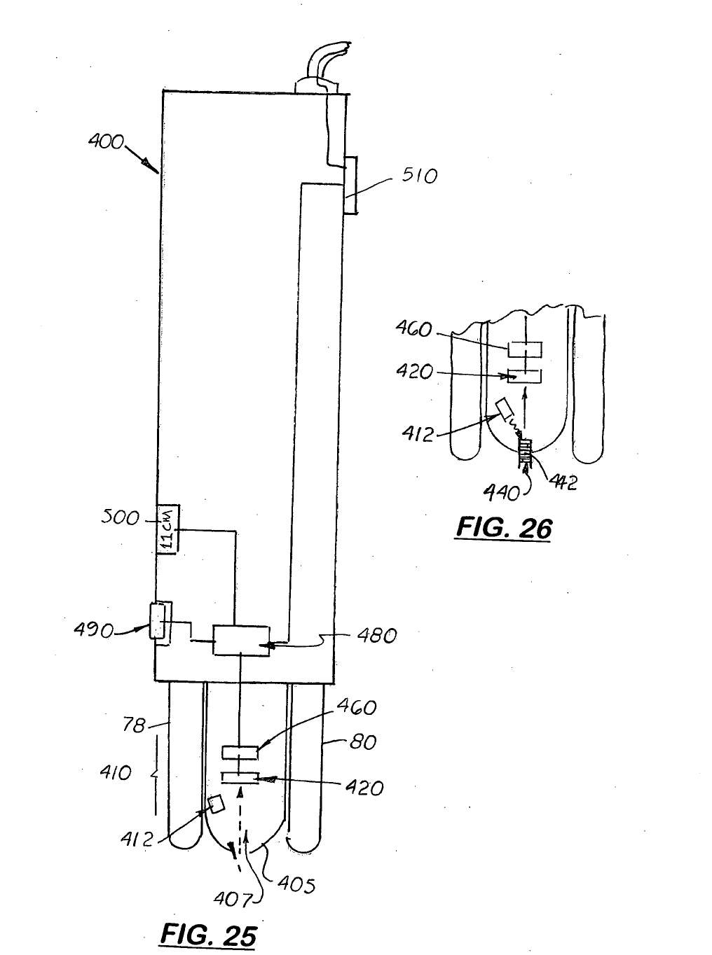

FIGS. 24 and 25 show another embodiment of the nerve stimulator, generally indicated by the reference number 400, with an optical measuring unit 410 built therein which is used to measure the linear distance the nerve stimulator 400 is moved across the surface. The nerve stimulator 400 includes a light emitter means 412 located inside a longitudinally aligned neck housing 405. In the preferred embodiment, the neck housing 405 is longitudinally aligned between the two probes 78, 80. The light emitter means 412 transmits light through an orifice 407 located at the tip of the neck housing 405. Located inside the neck housing 405 is a light receiver 420 that senses the light emitted from the light emitter means 412 and reflected from the skin surface. In an alternative embodiment shown in FIG. 25, a rolling ball 440 may be place between the orifice 407 and skin surface to create a more accurate reading. The roller ball 440 may include a lattice-shaped pattern 442 formed on its outer surface which has varying light reflecting characteristics, the variations in the light reflected from the rolling ball 440 can be easily sensed by the light receiver 420 when the rolling ball 440 rolls across the skin surface.

The light emitter means 412 may be a light emitting diode which has small power consumption and high light intensity. The light emitted from the light emitter means 412 is reflected off the skin surface or incident to the rolling ball 440 disposed at the lower tip portion of the neck housing 405.

Connected to the light receiver 420 is a conversion and output unit 460 that converts the variations in the light sensed by the light receiver 420 into an electrical signal and outputs the electrical signal. That is, when the sensor 400 is moved over the skin surface, light emitted from the light emitter means 412 is reflected from the surface or rolling ball 440 having the lattice-shaped pattern 442 continuously varies and the conversion and output unit 460 converts the variations in the light sensed by the light receiver 420 into an electrical signal and then outputs the electrical signal.

A calculation unit 480 is disposed inside the nerve sensor 400 and calculates the real distance using the electrical signal input from the conversion and output unit 460. The calculator unit 480 is also electrically connected to a LCD display 500 that indicated the distance measured. The calculator unit 480 is electrically connected to an ON/OFF switch 510.

The input button unit 490 is disposed inside the neck housing 405 and inputs a signal to the calculation unit 480 indicating that the orifice 407 or rolling ball 440 is positioned at the first point A or the second point B.

During operation, the user grasps the body of the nerve stimulator 400 and holds in vertically upright. The tip of the neck housing 405 or the rolling ball 440 is placed on the first point A, the input button unit 490 is pressed to indicate to the calculation unit 480 that the present position of the rolling ball 440 is the first point A. Then the nerve stimulator 400 is moved over the nerve path so that the orifice 407 or rolling ball 440 remains in contact with the skin. As the nerve stimulator 400 is moved, the light emitted through the orifice 407 and reflected off the skin or incident on the rolling ball 420 is sensed by the light receiver 420. The variation in the light sensed by the light receiver 420 is converted into an electrical signal that is output to the calculation unit 440. The nerve stimulator 400 is moved to the second point B. Then, when the orifice 407 or rolling ball 480 reaches the second point B, the input button unit 490 is pressed to indicate to the calculation unit 480 that the present position of the housing 10 is the second point B. Then, the calculation unit 480 recognizes the second point B and calculates a distance over which the orifice 407 or rolling ball 420 has rolled from the first point A to the second point B. The distance is then shown on the display 500.

In compliance with the statute, the invention described herein has been described in language more or less specific as to structural features. It should be understood however, that the invention is not limited to the specific features shown, since the means and construction shown is comprised only of the preferred embodiments for putting the invention into effect. The invention is therefore claimed in any of its forms or modifications within the legitimate and valid scope of the amended claims, appropriately interpreted in accordance with the doctrine of equivalents.

I claim:

1. A nerve conducting testing and measuring system, comprising:
a. a handheld nerve stimulator that includes an elongated body, an anode probe and a cathode probe longitudinally aligned and extending from one end of said body;
b. a linear skin measuring device attached to said stimulator, said linear skin distance measuring device includes a means for measuring the distance the anode and cathode probes are moved to a desired location on the skin or a nerve to be tested from the electrode sensors attached to the skin; and,
c. an electric nerve signal generator connected to said anode probe and said cathode probe on said handheld nerve simulator.

2. A nerve stimulator testing and measuring system, including:
a. a handheld nerve stimulator that includes an elongated body with an anode probe and a cathode probe longitudinally aligned and extending from one end thereof;
b. a linear skin measuring device;
c. means for attaching said linear skin measuring device to said anode or said cathode probes so that the distance said handheld nerve stimulator is moved along the skin from the reference electrode is measured; and,
d. an electric nerve signal generator connected to said anode probe and said cathode probe on said handheld nerve simulator.
